(19) United States
(12) Patent Application Publication
(10) Pub. No.: US 2007/0265352 A1
(43) Pub. Date: Nov. 15, 2007

(54) Synergistic mixtures of at least one 1,2 alkanediol such as 1,2-hexanediol and 1,2-octanediol with a further compound having antimicrobial properties

(76) Inventors: Joachim Roeding, Badenweiler (DE); Sabine Lange, Holzminden (DE); Gerhard Schmaus, Hoexter-Bosseborn (DE); Holger Joppe, Dassel (DE); Ravikumar Pillai, Emerson, NJ (US)

Correspondence Address: AKERMAN SENTERFITT, P.O. BOX 3188, WEST PALM BEACH, FL, 33402-3188, US

(21) Appl. No.: 11/254,346

(22) Filed: Oct. 20, 2005

Related U.S. Application Data
(63) Continuation-in-part of application No. 10/502,132, filed on Jul. 20, 2004.
(60) Provisional application No. 60/621,493, filed on Oct. 22, 2004.

(30) Foreign Application Priority Data
Feb. 19, 2002 (DE) DE 102 06 759.7

(51) Int. Cl. A61K 31/047; A01N 31/02; A01P 1/00; A61P 31/00; A23L 1/015

(52) U.S. Cl. 514/738; 426/335

(57) ABSTRACT

Antimicrobial active compositions and substances comprising at least one, preferably two, unbranched (straight-chain) 1,2-alkanediols and preferably a further preservative and also to products containing such mixtures in an antimicrobially effective quantity. One example of an antimicrobial mixture comprises: (a) 1,2-hexanediol and 1,2-octanediol and also optionally 1,2-pentanediol and/or 1,2-decanediol and (b) one, two or more substances selected from the group consisting of potassium sorbate, parabens and iodopropynyl butylcarbamate (IPBC).

### CROSS REFERENCE TO RELATED APPLICATIONS

This application is a Continuation-In-Part Application of U.S. application Ser. No. 10/502,132 filed on Jul. 20, 2004, pending, which is based upon German Patent Application No. DE 102 06 759.7 filed Feb. 19, 2002, and is a Non-Provisional Application of U.S. Provisional Application 60/621,493 filed Oct. 22, 2004, and which are hereby incorporated in their entirety by reference.

### FIELD OF THE INVENTION

The present invention relates to the field of antimicrobial active compositions and substances and, in particular, to certain mixtures, preparations and foodstuffs comprising at least one, preferably two, unbranched (straight-chain) 1,2-alkanediols and preferably a further preservative and also to products containing such mixtures in an antimicrobially effective quantity.

### BACKGROUND OF THE INVENTION

In the cosmetic and pharmaceutical industries and also in the food industry there is a constant demand for agents having antimicrobial properties, particularly for the preservation of otherwise perishable products (such as, for example cosmetics, pharmaceutical products or foodstuffs), but also for the immediate cosmetic or therapeutic treatment of micro-organisms that can have a detrimental influence on the human body or on the body of an animal. As an example, attention may be drawn to micro-organisms that can cause body odour, acne, mycoses or such like.

Although a large number of antimicrobial active substances are already employed in the technical fields under consideration, searches continue to be made for alternatives, in order to be able to implement selective special treatments and/or to be able to reduce side-effects. However, in the search for alternative agents having an antimicrobial and, in particular, a preserving effect it has to be borne in mind that the substances used in the cosmetic, pharmaceutical and/or foodstuff fields have to be:
toxicologically harmless,
highly cutaneously compatible,
stable (particularly in the conventional cosmetic and/or pharmaceutical formulations),
largely and preferably totally odourless and also
capable of being produced inexpensively (i.e. using standard processes and/or starting from standard precursors).

The search for suitable (active) substances that have one or more of the stated properties to a sufficient degree is rendered difficult for a person skilled in the art by virtue of the fact that there is no clear relationship between the chemical structure of a substance, on the one hand, and its biological activity in relation to certain micro-organisms (germs) and also its stability, on the other hand. Furthermore, there is no predictable connection between the antimicrobial effect, the toxicological harmlessness, the cutaneous compatibility and the stability of a substance.

### SUMMARY OF THE INVENTION

According to a first aspect, the invention relates to mixtures of
(a) at least one, preferably two, three or more straight-chain 1,2-alkanediols, the chain lengths of which (i) are different and (ii) in each case are in the range of 5 to 10 C-atoms, and
(b) at least one preservative selected from sorbate and its salts (e.g., potassium sorbate), parabene (methyl, ethyl, propyl and butyl parabens and mixtures), iodopropynyl butylcarbamate (IBPC; 3-iodo-2-propynyl-butylcarbamate), cosmetic chelating agents such as ethylene diamene tetraacetic acid (EDTA) and its salts (e.g., disodium EDTA, tetrasodium EDTA), tropolone compounds (e.g., tropolone (2-hydroxycyclohepta-2,4,6-trienone), thujaplicins (alpha-, beta- and/or gamma-thujaplicin)), and sisquiterpenes (e.g., bisabolol and farnesol).

In this context the proportions of the said diols and preservatives in the mixture are preferably set such that their antimicrobial action is synergistically intensified. The said 1,2-alkanediols include, in particular, 1,2-pentanediol, 1,2-hexanediol, 1,2-octanediol and 1,2-decanediol. In addition to the said 1,2-alkanediol mixtures, further (for example conventional) antimicrobial active compounds can be present, some of which—as explained further below—are able to develop a further synergistic action.

According to another aspect, the invention relates to antimicrobial mixtures comprising or consisting of:
(a) 1,2-hexanediol and 1,2-octanediol and also optionally 1,2-pentanediol and/or 1,2-decanediol and also
(b) one, two or more substances selected from the group consisting of potassium sorbate, parabens and iodopropynyl butylcarbamate (IPBC; 3-iodo-2-propynyl-butylcarbamate), an/or other microbial agent listed above.

In this connection the proportions of said diols in the mixture are preferably adjusted in such a way that their antimicrobial effect is synergistically intensified.

Parabens within the meaning of the present invention are C₁-C₄ esters of 4-hydroxybenzoic acid, in particular methylparaben, ethylparaben, n-propylparaben, n-butylparaben, isobutylparaben and mixtures thereof. Potassium sorbate is the potassium salt of sorbic acid (2,4-hexadienoic acid). Parabens can be used alone or in combination for preservation purposes especially in cosmetical and pharmaceutical preparations. The most common combination of parabens in these preparations is 0.18 wt. % methylparaben and 0.02 wt. % propylparaben.

The invention is based on the surprising perception that the mixtures according to the invention display a synergistically intensified antimicrobial effect at least in relation to selected germs, particularly in relation to Aspergillus niger, a mould fungus that can only be combated with difficulty.

In particular, it has become evident that the mixtures according to the invention can be used in outstanding manner in the form of an antimicrobial active-substance mixture, in particular for the preservation of otherwise perishable articles (see above).

Although specialists in the field had already been extensively concerned with the antimicrobial properties of 1,2-alkanediols, hitherto there had been no indication that mixtures consisting of (a) 1,2-hexanediol and 1,2-octanediol and also optionally 1,2-pentanediol and/or 1,2-decanediol and (b) at least one preservative chosen from the group consisting of potassium sorbate, parabens and iodopropynyl butylcarbamate (IPBC) have an antimicrobial action (at least in relation to selected germs) that is distinctly improved in the individual case.

WO 03/069994 describes synergistically effective mixtures of straight-chain, unbranched 1,2-alkanediols, for example of 1,2-hexanediol and 1,2-octanediol. The mixtures described therein display a good action in relation to Gram-negative bacteria such as Escherichia coli and Pseudomonas aeruginosa and in relation to yeasts such as Candida albicans. Although mixtures of 1,2-alkanediols according to WO 03/069994 also exhibit a good efficacy in relation to fungi such as Aspergillus niger, the efficacy is not totally satisfactory. Although a combination of 0.25 wt. % 1,2-hexanediol, 0.25 wt. % 1,2-octanediol and 0.05 wt. % Euxyl K 400 (Euxyl K 400 is a mixture consisting of 80 wt. % 2-phenoxyethanol and 20 wt. % 1,2-dibromo-2,4-dicyanobutane) exhibited an improved efficacy in relation to Aspergillus niger, the germ was not totally eliminated even after a duration of action of 28 days.

EP 1 206 933 relates to mixtures of 1,2-octanediol or an analogue and a preservative, in particular iodopropynyl butylcarbamate. This document, however, does not disclose a mixture comprising 1,2-hexanediol, 1,2-octanediol and a further preservative.

Studies relating to the efficacy of the disclosed mixtures in relation to Aspergillus niger are likewise not disclosed.

With regard to mixtures of 1,2-alkanediols, a deficiency in their efficacy in the case of mould fungi (e.g. the “problem germ” Aspergillus niger) is accordingly to be noted. For the complete inhibition of mould fungi, therefore, high usage concentrations of mixtures consisting of 1,2-alkanediols have been necessary hitherto.

It was now particularly surprising that the mixtures according to the invention display a strongly synergistic efficacy and are distinctly superior to the individually metered preservatives potassium sorbate, parabens and iodopropynyl butylcarbamate or to a mixture of 1,2-hexanediol and 1,2-octanediol at the same concentration, particularly with regard to the germ-count reduction. In particular, in the individual case a CFU value (CFU=number of colony-forming units) of 0 was attained only with said mixtures according to the invention.

### DETAILED DESCRIPTION OF THE INVENTION

EDTA and its salts (e.g. Disodium EDTA and Tetrasodium EDTA) are used often in topical personal care products as chelating agents. It is also known that these chelating agents have antimicrobial activity, though the activity is very weak.

Tropolone compounds are known to possess anti-inflammatory and antimicrobial properties.

Sesquiterpenes like bisabolol and famesol are used in personal care products as anti-inflammatory and antiaging ingredients, respectively. They are also known to have antimicrobial properties.

The present disclosure describes enhanced/synergistic antimicrobial activity of mixtures and cosmetic compositions comprising at least one alkane diol and at least one compound selected from the group of tropolone-compounds (in particular tropolone and thujaplicins), chelating agents (in particular EDTA and its sodium salts) and sesquiterpenes (in particular bisabolol and farnseol).

Advantageous are mixtures and cosmetic compositions containing at least one alkane diol and one or more chelating agents (particularly EDTA, Disodium EDTA, Tetrasodium EDTA). Also advantageous are mixtures and cosmetic compositions containing more than one alkane diol and one or more sesquiterpenes, in particular bisabolol (preferably alpha-bisabolol). Preferred are mixtures and cosmetic compositions containing two or more alkane diols and at least one chelating agent and at least one sesquiterpene. Another preferred embodiment relates to mixtures and cosmetic compositions comprising at least one, preferably two or more, alkane diols and at least one tropolone-compound, in particular tropolone (2-hydroxycyclohepta-2,4,6-trienone) and/or one or more thujaplicins (alpha-,beta-and/or gamma-thujaplicin).

Preferred alkane diols have a total of 3 to 12 carbon atoms, preferably 6 to 10 carbon atoms. Advantageous alkane diols are propylene glycol, 2-methyl-propane-1,3-diol (methyl propanediol), butylene glycol, 1,6-hexane diol, 2-methylpentane-2,4-diol (hexylene glycol), 2,5-hexane diol, 3,6-octane diol, 2-ethyl-1,3-hexane diol, 1,3-hexane diol, 1,3-octane diol, 1,3-decane diol, 1,2-pentane diol, 1,2-hexane diol, 1,2-octane diol, 1,2-decane diol and mixtures thereof.

Preferred alkane diols are straight chain diols. Preferred 1,2-alkane diols are selected from the group consisting of 1,2-hexane diol, 1,2-octane diol, 1,2-decane diol and binary or ternary mixtures of these three diols. The weight ration in most preferred mixtures of 1,2-hexane diol and 1,2-octane diol are in the range of 10:1 to 1:10, preferred in the range of 3:1 to 1:3, and particularly preferred in the range of 2:1 to 1:2.

Tests using mixtures and cosmetic compositions of the current disclosure reveal that alkane diols like 1,2-hexane diol or and 1,2-octane diol (caprylyl glycol) and mixtures of 1,2-hexane diol and 1,2-octane diol can significantly increase the anti-microbial activity of tropolone-compounds like tropolone, chelating agents like EDTA and sesquiterpenes like bisabolol, well enough to effectively preserve said mixtures and compositions. This effect is in particular surprising with respect to Aspergillus niger, a problematic fungus, which plays an important role in the preservation of cosmetic products.

The cosmetic compositions of the present disclosure may contain 1,2-hexanediol, 1,2-octanediol and a tropolone-compound, a chelating agent and/or a sesquiterpene. The cosmetic compositions will also contain other components like solvents, emulsifiers, glycerides, fats, pigments, UV-filters and other ingredients that can be added to a topical formulation. The compositions may further contain preservatives.

Particularly preferred are mixtures according to the invention comprising (a) 1,2-hexanediol and 1,2-octanediol and also optionally 1,2-pentanediol and/or 1,2-decanediol and also (b) iodopropynyl butylcarbamate (IPBC). By reason of their particularly significant intensification of effect, such mixtures are suitable, in particular, for the purpose of combating Aspergillus niger, even at low dosage of the mixture according to the invention. To a mixture of (a) 1,2-hexanediol and 1,2-octanediol and also optionally 1,2-pentanediol and/or 1,2-decanediol only a small proportion of IPBC, for example within the range of 2-10 wt. %, for example merely 5 wt. %, relative to portion (a), needs to be added in order to bring about a complete reduction of the germ count of Aspergillus niger. In the case of a usage quantity of 0.4 wt. % of 1,2-hexanediol and 1,2-octanediol and also optionally 1,2-pentanediol and/or 1,2-decanediol, this corresponds, for example, to a quantity of IPBC amounting to just under 0.02 wt. %, in each instance relative to the total weight of the end product.

Relative to the total mass of the 1,2-alkanediols to be employed in accordance with the invention, the proportion of 1,2-hexanediol and 1,2-octanediol preferably lies in each instance within the range from 10 wt. % to 90 wt. %, but preferably in each instance within the range from 20 wt. % to 80 wt. %.

The mixtures according to the invention are not only suitable for the preservation of perishable products such as, for example, cosmetic products, pharmaceutical products or foodstuffs but, by reason of their synergistically intensified antimicrobial efficacy, may also be employed
(a) for the cosmetic treatment of micro-organisms causing body odour,
(b) for the cosmetic treatment of micro-organisms causing acne,
(c) for the cosmetic treatment of micro-organisms causing mycoses and
(d) for the treatment of micro-organisms on or in inanimate matter.

The mixtures according to the invention display their synergistic action in relation to a large number of Gram-positive bacteria, Gram-negative bacteria, mould fungi and yeasts. There is a particularly good effect in relation to Gram-negative bacteria such as Escherichia coli and Pseudomonas aeruginosa, in relation to yeasts such as Candida albicans and precisely—as already mentioned—in relation to fungi such as Aspergillus niger. In this connection the very good efficacy of the mixtures according to the invention in relation to Aspergillus niger, a mould fungus that can only be combated with great difficulty, is to be regarded as particularly advantageous.

The present invention also relates to the use of a mixture comprising or consisting of:
(a) 1,2-hexanediol and 1,2-octanediol and also optionally 1,2-pentanediol and/or 1,2-decanediol and also
(b) one, two or more substances selected from the group consisting of potassium sorbate, parabens and iodopropynyl butylcarbamate (IPBC)
by way of antimicrobial active-substance mixture.

The present invention relates, in addition, to appropriate processes for the cosmetic and/or therapeutic treatment of germs, in particular of (a) micro-organisms causing body odour, (b) micro-organisms causing acne and/or (c) micro-organisms causing mycoses, comprising the topical application of an antimicrobially effective quantity of a mixture according to the invention, the proportions of said diols in the mixture preferably being adjusted in such a way that their antimicrobial effect is synergistically intensified.

Preferred configurations of the processes according to the invention correspond to the preferred configurations of the use according to the invention that have been elucidated above.

The amount of alkane diol(s) typically is from 0.1 to 10%, preferably 0.4 to 5.0%, based on the total weight of the cosmetic composition.

The amount of chelating agent typically is from 0.01 to 1%, preferably 0.1 to 0.5%, based on the total weight of the cosmetic composition.

The amount of the sesquiterpenes typically is from 0.01 to 1%, preferably 0.1 to 0.5%, based on the total weight of the cosmetic composition.

The amount of the tropolone compounds typically is from 0.0001 to 10%, preferably 0.01 to 4%, based on the total weight of the cosmetic composition.

The amount of potassium sorbate, in particular, is generally in the range of 0.01 to 0.6%, preferably 0.05-0.4%, particularly preferably 0.1-0.2%, based on the total weight of the cosmetic composition.

The maximum amount of any one parabene is 0.4%, and the maximum amount of any mixture of parabenes is 0.8%. Preferably the amount of paraben(s) is in the range of 0.01 to 0.4%, more preferably 0.05-0.3%, particularly preferably 0.1-0.2%, based on the total weight of the cosmetic composition.

In the case of use of combinations of different preservatives, limitations are based generally on human tolerance (rash, irritation, inflammation) depending upon individual desired product (topical composition, food product, etc).

The human skin is colonised by a large number of different micro-organisms, including the micro-organisms already named above, as well as others. Most of these micro-organisms are not pathogenic and are irrelevant to the physiological condition of the skin and to the odour thereof. Others, on the other hand, can have a crucial influence on the healthy condition of the skin.

As some studies have now shown, the synergistically effective mixtures according to the invention are highly effective not only in relation to the germs already described above but also in relation to Staphylococcus epidermidis, Corynebacterium xerosis, Brevibacterium epidermidis, Propionibacterium acnes and also in relation to Trichophyton and Epidermophyton species, so that they can also be employed as agents for the treatment (combating) of armpit odour and foot odour or body odour in general, as agents for combating acne, as antidandruff agents and for the treatment of mycoses (in particular, dermatomycoses).

The term “treatment” within the scope of the present text is understood to mean any form of exerting influence on the micro-organisms in question, in the course of which the propagation of these micro-organisms is inhibited and/or the micro-organisms are killed.

The usage concentration of the mixtures according to the invention in the case where they are used as preservative or as antimicrobial active substance in a foodstuff or in a cosmetic or pharmaceutical preparation preferably lies within the range from 0.01 wt. % to 30 wt. %, but particularly preferably within the range from 0.1 wt. % to 5 wt. %, in each instance relative to the total mass of the foodstuff or of the preparation. The foodstuff or preparation comprises, in addition, conventional further constituents, see below. The respective proportion of the (a) 1,2-diols and/or (b) preservatives to be used in accordance with the invention in mixtures according to the invention may lie below the antimicrobially effective quantity, considered separately, if the available total quantity of these substances is sufficiently high in order to obtain an antimicrobial action of the overall mixture. This holds, in particular, for the action in relation to Aspergillis niger.

In a preferred process according to the invention for the cosmetic and/or therapeutic treatment of (a) micro-organisms causing body odour, (b) micro-organisms causing acne and/or (c) micro-organisms causing mycoses the usage concentration of the synergistically effective mixtures according to the invention also lies within the range between 0.01 wt. % and 30 wt. % and, particularly preferably, within the range between 0.1 wt. % and 5 wt. %, in each instance relative to the total mass of the cosmetic or pharmaceutical product that comprises the mixture.

The synergistically effective mixtures may in this connection find application (a) prophylactically or (b) when required.

The concentration of the quantity of active substance which, for example, is to be applied daily is variable and depends on the physiological condition of the test subject and also on individual-specific parameters such as age or body weight. The synergistically effective mixtures according to the invention may find application both on their own and also in combination with further antimicrobially active substances.

Attention is drawn to the fact that within the scope of the present text the 1,2-alkanediols to be employed in accordance with the invention may be present both as an appropriate 2S-configured enantiomer and as a 2R-configured enantiomer and also in the form of arbitrary mixtures of these 2S-configured and 2R-configured enantiomers. For commercial reasons, although it is particularly advantageous to employ mixtures of racemates of the respective 1,2-alkanediols to be employed in accordance with the invention for the purpose of combating micro-organisms, since these are particularly readily accessible synthetically, the pure enantiomers or non-racemic mixtures of these enantiomers are also suitable for the purposes according to the invention.

Further uses/processes and mixtures/compositions according to the invention can be inferred from the following implementations and from the appended Claims.

Compositions that contain a mixture according to the invention, in particular to the extent that they are employed against germs causing body odour, are applied, as a rule, topically in the form of solutions, creams, lotions, gels, sprays or such like. For other purposes, in many cases an oral (tablets, capsules, powders, drops), intravenous, intra-ocular, intraperitoneal or intramuscular application or an application in the form of an impregnated dressing is sensible.

The mixtures according to the invention can be incorporated without difficulty into marketable cosmetic or dermatological formulations such as, inter alia, pump sprays, aerosol sprays, creams, ointments, tinctures, lotions, nail-care products (e.g. nail varnishes, nail-varnish removers, nail balms) and such like. In this connection it is also possible, and in many cases advantageous, to combine the synergistic mixtures according to the invention with further active substances, for example with other antimicrobially, antimycotically or antivirally active substances. In this connection the cosmetic and/or dermatological/keratological formulations containing the synergistic mixtures according to the invention may otherwise be composed as usual and may serve for treating the skin and/or the hair along the lines of a dermatological treatment or along the lines of a treatment within the field of grooming cosmetics. But they may also be employed in make-up products in the field of decorative cosmetics.

If the mixtures according to the invention are employed as active substances for the preservation of organic material then a further preservative or several further preservatives may advantageously be employed in addition. Preferably chosen in this connection are preservatives such as benzoic acid, its esters and salts, propionic acid and its salts, salicylic acid and its salts, formaldehyde and paraformaldehyde, 2-hydroxybiphenyl ether and its salts, 2-zinc sulfidopyridine-N-oxide, inorganic sulfites and bisulfites, sodium iodate, chlorobutanol, 4-ethylmercury(II)-5-amino-1,3-bis(2-hydroxybenzoic acid), its salts and esters, dehydroacetic acid, formic acid, 1,6-bis(4-amidino-2-bromophenoxy)-n-hexane and its salts, the sodium salt of ethylmercury(II)-thiosalicylic acid, phenylmercury and its salts, 10-undecylenic acid and its salts, 5-amino-1,3-bis(2-ethylhexyl)-5-methylhexahydropyrimidine, 5-bromo-5-nitro-1,3-dioxan, 2-bromo-2-nitro-1,3-propanediol, 2,4-dichlorobenzyl alcohol, N-(4-chlorophenyl)-N′-(3,4-dichlorophenyl)urea, 4-chloro-m-cresol, 2,4,4′-trichloro-2′-hydroxydiphenyl ether, 4-chloro-3,5-dimethylphenol, 1,1′-methylene-bis(3-(1-hydroxymethyl-2,4-dioximidazolidine-5-yl)urea), poly(hexamethylene biguanide)hydrochloride, 2-phenoxyethanol, hexamethylenetetramine, 1-(3-chloroallyl)-3,5,7-triaza-1-azonia adamantane chloride, 1(4-chlorophenoxy)1(1H-imidazole-1-yl)-3,3-dimethyl-2-butanone, 1,3-bis(hydroxymethyl)-5,5-dimethyl-2,4-imidazolidinedione, benzyl alcohol, Octopirox, 1,2-dibromo-2,4-dicyanobutane, 2,2′-methylene-bis(6-bromo-4-chlorophenol), bromochlorophene, mixture of 5-chloro-2-methyl-3(2H)isothiazolinone and 2-methyl-3(2H)isothiazolinone with magnesium chloride and magnesium nitrate, 2-benzyl-4-chlorophenol, 2-chloroacetamide, chlorhexidine, chlorhexidine acetate, chlorhexidine gluconate, chlorhexidine hydrochloride, 1-phenoxypropane-2-ol, N-alkyl(C₁₂-C₂₂)trimethylammonium bromide and chloride, 4,4-dimethyl-1,3-oxazolidine, N-hydroxymethyl-N-(1,3-di(hydroxymethyl)-2,5-dioxoimidazolidine-4-yl)-N′-hydroxymethylurea, 1,6-bis(4-amidinophenoxy)-n-hexane and its salts, glutaraldehyde, 5-ethyl-1-aza-3,7-dioxabicyclo(3.3.0)octane, 3-(4-chlorophenoxy)-1,2-propanediol, Hyamine, alkyl-(C₈-C₁₈) -dimethylbenzylammonium chloride, alkyl-(C₈-C₁₈) -dimethylbenzylammonium bromide, alkyl-(C₈-C₁₈)-dimethylbenzylammonium saccharinate, benzyl hemiformal, sodium hydroxymethyl aminoacetate or sodium hydroxymethyl aminoacetate.

If the mixtures according to the invention are to be employed chiefly for the purpose of inhibiting the growth of undesirable micro-organisms on or in animal organisms, here too a combination with further antibacterially or antimycotically active substances is advantageous in many cases. Worth mentioning in this respect as further active substances, in addition to the large group of classical antibiotics, are, in particular, the products that are relevant to cosmetics, such as triclosan, climbazole, octoxyglycerin, Octopirox(1-hydroxy-4-methyl-6-(2,4,4-trimethylpentyl)-2(1H)-pyridone, 2-aminoethanol), chitosan, farnesol, glycerol monolaurate or combinations of the stated substances, which are employed, inter alia, for countering armpit odour, foot odour or scaling.

The mixtures according to the invention may advantageously be combined, particularly in cosmetic preparations, with auxiliary substances such as, for example:

Further preservatives, further antimicrobial agents such as, for example, further antibacterial agents or fungicides, abrasives, anti-acne agents, agents for countering ageing of the skin, anticellulitis agents, antidandruff agents, anti-inflammatory agents, irritation-preventing agents, irritation-inhibiting agents, anti-oxidants, astringents, antihidrotics, antiseptic agents, antistatic agents, binders, buffers, excipient materials, chelating agents, cell stimulants, cleansing agents, grooming agents, depilatories, surface-active substances, deodorising agents, antiperspirants, emollients, emulsifiers, enzymes, essential oils, fibres, film-formers, fixatives, foaming agents, foam stabilisers, substances for preventing foaming, foam boosters, gelling agents, gel-forming agents, hair-care agents, hair-deforming agents, hair-smoothing agents, moisture-donating agents, moistening substances, moisture-retaining substances, bleaching agents, starching agents, stain-removing agents, optical brightening agents, impregnating agents, dirt-repelling agents, friction-reducing agents, lubricants, moisturising creams, ointments, opacifying agents, plasticising agents, covering agents, polish, lustring agents, polymers, powders, proteins, regreasing agents, abrading agents, silicones, skin-soothing agents, skin-cleansing agents, skin-care agents, skin-healing agents, skin-lightening agents, skin-protecting agents, skin-softening agents, cooling agents, skin-cooling agents, warming agents, skin-warming agents, stabilisers, UV-absorbing agents, UV filters, detergents, softeners, suspending agents, skin-tanning agents, thickening agents, vitamins, oils, waxes, fats, phospholipids, saturated fatty acids, mono-unsaturated or polyunsaturated fatty acids, a-hydroxy acids, polyhydroxy fatty acids, liquefiers, dyestuffs, paint-protecting agents, pigments, anticorrosives, aromas, flavouring substances, odoriferous substances, polyols, surfactants, electrolytes, organic solvents or silicone derivatives.

Furthermore, the mixtures according to the invention may also be employed particularly advantageously in combination with antihidrotic active substances (antiperspirants) for the purpose of combating body odour. Aluminium salts such as aluminium chloride, aluminium chlorohydrate, aluminium nitrate, aluminium sulfate, aluminium acetate etc. find application above all by way of antihidriotic active substances. But, in addition to these, the use of zinc compounds, magnesium compounds and zirconium compounds may also be advantageous. For practical use in cosmetic and dermatological antiperspirants, substantially the aluminium salts and—to a somewhat lesser degree—aluminium/zirconium-salt combinations have proved worthwhile. Worth mentioning in addition to these are the partially neutralised. and hence more cutaneously compatible but not quite so effective, aluminium hydroxychlorides.

If the mixtures according to the invention are to be employed for the antimicrobial treatment of a surface (e.g. of a human body or the body of an animal), then in many cases a combination with (metal) chelating agents is advantageous. (Metal) chelating agents to be preferentially employed in this connection are, inter alia, α-hydroxy fatty acids, phytinic acid, lactoferrin, α-hydroxy acids such as, inter alia, citric acid, lactic acid and malic acid and also humic acids, bile acids, bile extracts, bilirubin, biliverdin or EDTA, EGTA and derivatives thereof.

For practical use, the cosmetic and/or dermatologically effective mixtures according to the invention are applied in sufficient quantity onto the skin and/or onto the hair in the conventional manner for cosmetics and dermatics. Particular advantages in this regard are offered by cosmetic and dermatological preparations that contain a mixture according to the invention and that additionally act as sunscreen agents. These preparations advantageously contain at least one UVA filter and/or at least one UVB filter and/or at least one inorganic pigment. In this case the preparations may be present in various forms, such as are conventionally employed, for example, for sunscreen preparations. Thus they may be, for example, a solution, an emulsion of the water-in-oil (W/O) type or of the oil-in-water (O/W) type, or a multiple emulsion, for example of the water-in-oil-in-water (W/O/W) type, a gel, a hydrodispersion, a solid stick or even an aerosol.

As mentioned, preparations that contain a mixture according to the invention may advantageously be combined with substances that absorb UV radiation, the total quantity of the filter substances amounting to, for example, 0.01 wt. % to 40 wt. %, preferably 0.1 wt. % to 10 wt. %, in particular 1.0 wt. % to 5.0 wt. %, relative to the total weight of the preparations, in order to make cosmetic preparations available that protect the hair or the skin from ultraviolet radiation.

In formulations containing mixtures according to the invention for the topical prophylactic or cosmetic treatment of the skin a high proportion of grooming substances is normally advantageous. According to a preferred embodiment, the compositions contain one or more grooming, animal and/or vegetable, fats and oils such as olive oil, sunflower oil, refined soybean oil, palm oil, sesame oil, rapeseed oil, almond oil, borage oil, evening-primrose oil, copra oil, shea butter, jojoba oil, sperm oil, beef tallow, neat's-foot oil and lard and also optionally further grooming constituents such as, for example, fatty alcohols with 8-30 C-atoms.

Grooming substances that can be combined excellently with the synergistic mixtures according to the invention furthermore also include
ceramides, the term ‘ceramides’ being understood to mean N-acylsphingosines (fatty acid amides of sphingosine) or synthetic analogues of such lipids (so-called pseudo-ceramides) which clearly improve the water-retaining capacity of the horny layer
phospholipids, for example soya lecithin, egg lecithin and cephalins
Vaseline, paraffin oils and silicone oils; the latter include, inter alia, dialkylaryl siloxanes and alkylaryl siloxanes such as dimethyl polysiloxane and methyl phenyl polysiloxane and also the alkoxylated and quaternised derivatives thereof.

Cosmetic preparations that contain mixtures according to the invention may also contain anti-oxidants, in which case use may be made of all the anti-oxidants that are suitable or customary for cosmetic and/or dermatological applications.

Cosmetic preparations that contain mixtures according to the invention may also contain vitamins and vitamin precursors, in which case use may be made of all the vitamins and vitamin precursors that are suitable or customary for cosmetic and/or dermatological applications. Worth mentioning here are, in particular, vitamins and vitamin precursors such as tocopherols, vitamin A, nicotinic acid and nicotinamide, further vitamins of the B-complex, in particular biotin and vitamin C, panthenol and derivatives thereof, in particular the esters and ethers of panthenol and also cationically derivatised panthenols such as, for example, panthenol triacetate, panthenol monoethyl ether and the monoacetate thereof and also cationic panthenol derivatives.

Cosmetic preparations that contain mixtures according to the invention may also contain anti-inflammatory or anti-erythematic or antipruritic active substances. In this connection, use may be made of all the anti-inflammatory or anti-erythematic or antipruritic active substances that are suitable or customary for cosmetic and/or dermatological applications.

Cosmetic preparations that contain mixtures according to the invention may also contain active substances having a skin-lightening or skin-tanning action. According to the invention, in this connection use may be made of all the skin-lightening or skin-tanning active substances that are suitable or customary for cosmetic and/or dermatological applications.

Cosmetic preparations that contain mixtures according to the invention may also contain anionic, cationic, non-ionic and/or amphoteric surfactants, particularly if crystalline or microcrystalline solids, for example inorganic micropigments, are to be incorporated into the preparations.

The invention will be elucidated in more detail below on the basis of the following example. Unless otherwise stated, the figures relate to the weight.

### EXAMPLE 1

### Comparison in the Test for Sufficient Preservation by (i) a Mixture Consisting of 1,2-hexanediol and 1,2-octanediol and (ii) Mixtures According to the Invention

A test for sufficient preservation was carried out in accordance with the European Pharmacopoeia.

The test consequently consists of the contamination of the preparation, if possible in its final ratio, with a prescribed inoculum of suitable micro-organisms, of the storage of the inoculated preparation at a certain temperature, of the taking of samples from the container at certain time-intervals, and of the determination of the number of micro-organisms in the samples so taken. The preserving properties are sufficient if, under the conditions of the test, an unambiguous diminution—or, where appropriate, no increase—of the germ count in the inoculated preparations results after the prescribed times at the prescribed temperatures. Experimental details of the test procedure are described in the European Pharmacopoeia (ISBN 3-7692-2768-9; Supplement 2001 to the 3^{rd} Edition, pages 421-422, Chapter 5.1.3).

Test germs:
The following micro-organism strains were used for the tests for sufficient preservation:
A: Escherichia coli ATCC 8739
B: Pseudomonas aeruginosa ATCC 9027
C: Staphylococcus aureus ATCC 6538
D: Candida albicans ATCC 10231
E: Aspergillus niger ATCC 16404
The initial germ count (CFU/g; “0 value”) in the different series of tests was within the range from 250 000 to 320 000.

Formulation:
For the tests for sufficient preservation, mixtures according to the invention (active-substance combinations B-D, see below) were incorporated into separate O/W emulsions, in defined quantity in each instance. For comparison purposes, a mixture of 1,2-hexanediol and 1,2-octanediol (active substance A) was incorporated into a separate O/W emulsion.

Formulation of the O/W Emulsion Used:

| | INCI Name | Manufacturer | Wt. % |
|---|---|---|---|
| Phase A | | | |
| Dracorin CE | Glyceryl | Symrise | 4.0 |
| 614035 | Stearate Citrate | | |
| PCL Solid | Stearyl | Symrise | 3.0 |
| 660086 | Heptanoate, | | |
| | Stearyl | | |
| | Caprylate | | |
| Paraffinöl ° E | Paraffinum | Parafluid | 7.0 |
| | Liquidum | | |
| Lanette 18 | Stearyl Alcohol | Cognis | 1.5 |
| Dracorin GMS | Glyceryl | Symrise | 1.5 |
| 647834 | Stearate | | |
| Dow Corning 200 | Dimethicone | Dow | 2.0 |
| fluid | | Corning | |
| Phase B | | | |
| water, | Water (aqua) | | 80.75 |
| demineralised | | | |
| Carbopol ETD 2050 | Carbomer | Noveon | 0.15 |
| polymer | | | |
| Phase C | | | |
| Neutralizer AMP- | Amino | Dow/Angus | 0.1 |
| 95 | Methylpropanol | | |
| Total: | | | 100.0 |

pH value: 5.5

Result:

The results of the preservative-loading tests relating to the active-substance combinations studied, consisting of mixtures according to the invention and of the reference system 1,2-hexanediol/1,2-octanediol (active substance A; Table A), are represented in Tables A to D. Here the synergistic effect of mixtures according to the invention is shown, above all, in the residual germ counts for Aspergillus niger remaining after 28 days. As is evident from Tables B to D, in the case of Aspergillus niger, a particularly problematic germ with regard to the preservation of industrial products, the germ count was able to be reduced to 0 within 28 days by using mixtures according to the invention. On the other hand, active substance A (1,2-hexanediol+1,2-octanediol; quantitative ratio 1:1), which was tested for comparison purposes in a dosage of 0.5 wt. %, enabled no such significant reduction of the number of colony-forming units (CFU) in the case of Aspergillus niger. This series of tests consequently shows in exemplary manner that active-substance mixtures consisting of (a) 1,2-hexanediol, 1,2-octanediol and optionally 1,2-pentanediol and/or 1,2-decanediol and (b) a further preservative selected from the group comprising potassium sorbate, parabens and IPBC may have a synergistic, further improved effect. Tables A-D: Test for sufficient preservation in respect of (i) an active-substance combination A consisting of 1,2-hexanediol and 1,2-octanediol (reference) and (ii) mixtures according to the invention having an intensified synergistic effect (active-substance combinations B-D); [CFU/ml]

TABLE A
Active substance A: 1,2-hexanediol and 1,2-octanediol (weight ratio 1:1)
O/W emulsion with 0.5% active
substance A

| | Escherichia | Pseudomonas | Staphylococcus | Candida | Aspergillus |
|---|---|---|---|---|---|
| Days | coli | aeruginosa | aureus | albicans | niger |
| 0 | 300 000 | 320 000 | 310 000 | 310 000 | 280 000 |
| 2 | 0 | 0 | 100 | 30 000 | 160 000 |
| 7 | 0 | 0 | 0 | 0 | 100 000 |
| 28 | 0 | 0 | 0 | 0 | 14 000 |

TABLE B
Active substance B: active substance A and potassium
sorbate in a weight ratio of 2:1
O/W emulsion with 0.4% active
substance A and 0.2% potassium sorbate

| | Escherichia | Pseudomonas | Staphylococcus | Candida | Aspergillus |
|---|---|---|---|---|---|
| Days | coli | aeruginosa | aureus | albicans | niger |
| 0 | 270 000 | 310 000 | 310 000 | 300 000 | 250 000 |
| 2 | 0 | 0 | 0 | 0 | 0 |
| 7 | 0 | 0 | 0 | 0 | 0 |
| 28 | 0 | 0 | 0 | 0 | 0 |

TABLE C
Active substance C: active substance A and parabens (mixture
consisting of methyl, ethyl, propyl and butyl parabens) in a weight ratio of 2:1
O/W emulsion with 0.4% active
substance A and 0.2% parabens

| | Escherichia | Pseudomonas | Staphylococcus | Candida | Aspergillus |
|---|---|---|---|---|---|
| Days | coli | aeruginosa | aureus | albicans | niger |
| 0 | 270 000 | 310 000 | 310 000 | 300 000 | 250 000 |
| 2 | 0 | 0 | 0 | 0 | 0 |
| 7 | 0 | 0 | 0 | 0 | 0 |
| 28 | 0 | 0 | 0 | 0 | 0 |

TABLE D
Active substance D: active substance A and IPBC in a weight ratio of 20:1
O/W emulsion with 0.4% active
substance A and 0.02% IPBC

| | | Pseudomonas | Staphylococcus | Candida | Aspergillus |
|---|---|---|---|---|---|
| Days | Escherichia coli | aeruginosa | Aureus | albicans | niger |
| 0 | 270 000 | 310 000 | 310 000 | 300 000 | 250 000 |
| 2 | 0 | 0 | 0 | 34 000 | 200 |
| 7 | 0 | 0 | 0 | 100 | 0 |
| 28 | 0 | 0 | 0 | 0 | 0 |

The synergistic intensification of the efficacy of the active-substance combinations according to the invention can also be documented on the basis of the available data with the aid of Kull's equation (F. C. Kull et al.; Applied Microbiology Vol. 9, pp. 538-541 (1961); David C. Steinberg; Cosmetics & Toiletries Vol. 115 (No. 11), pp. 59-62; November 2000).

Cosmetic compositions relating to mixtures of (a) at least one, preferably two, three or more straight-chain 1,2-alkanediols, the chain lengths of which (i) are different and (ii) in each case are in the range of 5 to 10 C-atoms, and (b) at least one preservative, are illustrated by the following Examples 2-5. Unless indicated otherwise, all percentages are in % by weight.

### EXAMPLE 2

| | Formula 1 | Formula 2 |
|---|---|---|
| Raw Material | % Weight | % Weight |
| Phase 1 | | |
| Water | 98.80% | 97.60% |
| Carbomer | 0.60% | 0.60% |
| Phase 2 | | |
| Disodium EDTA | 0.10% | 0.10% |
| 1,2-hexane diol and 1,2-octane diol | | 1.20% |
| (1:1 w/w) | | |
| Phase 3 | | |
| Aminomethyl Propanol | 0.50% | 0.50% |

### EXAMPLE 3

| | Formula 3 | Formula 4 |
|---|---|---|
| Raw Material | % weight | % weight |
| Phase 1 | | |
| Glyceryl Stearate Citrate | 4.00% | 4.00% |
| Glyceryl Stearate | 1.50% | 1.50% |
| Stearyl alcohol | 1.50% | 1.50% |
| Stearyl Heptanoate & Stearyl Caprylate | 3.00% | 3.00% |
| Mineral oil | 6.00% | 6.00% |
| Bisabolol | 0.20% | 0.20% |
| Cyclopentasiloxane & Cyclohexasiloxane | 2.00% | 2.00% |
| Phase 2 | | |
| Water | 81.55% | 80.55% |
| Carbomer | 0.15% | 0.15% |
| Phase 3 | | |
| Aminomethyl Propanol | 0.10% | 0.10% |
| Phase 4 | | |
| 1,2-hexane diol and 1,2-octane diol | | 1.00% |
| (1:1 w/w) | | |

### EXAMPLE 4

| | Formula 5 | Formula 6 |
|---|---|---|
| Raw Material | % weight | % weight |
| Phase 1 | | |
| Glyceryl Stearate Citrate | 4.00% | 4.00% |
| Glyceryl Sterate | 1.50% | 1.50% |
| Stearyl alcohol | 1.50% | 1.50% |
| Stearyl Heptanoate & Stearyl Caprylate | 3.00% | 3.00% |
| Mineral Oil | 6.00% | 6.00% |
| (−)-alpha-Bisabolol | 0.10% | 0.10% |
| Cyclopentasiloxane & Cyclohexasiloxane | 2.00% | 2.00% |
| Phase 2 | | |
| Water | 80.55% | 80.85% |
| Disodium EDTA | 0.10% | 0.10% |
| Carbomer | 0.15% | 0.15% |
| Phase 3 | | |
| Aminomethyl Propanol | 0.10% | 0.10% |
| Phase 4 | | |
| 1,2-hexane diol and 1,2-octane diol | | 0.80% |
| (1:1 w/w) | | |

The following microorganisms were used for the challenge tests:
EC: Escherichia coli
PA: Pseudomonas aeruginosa
SA: Staphylococcus aureus
CA: Candida albicans
AN: Aspergillus niger

The challenge test results for the cosmetic formulations of Example 3 (all having a pH-value 5.5) were as follows:

| | Microbial Count, CFU/ml | | | | | | |
|---|---|---|---|---|---|---|---|
| | 2 days | | | | | 7 days | 28 days |
| Sample | EC | PA | SA | CA | AN | AN | AN |
| Formula 4 | 0 | 0 | 0 | 0 | 60,000 | 13,000 | 100 |
| Formula 3 | 287,000 | 194,000 | 60,000 | 460,000 | 208,000 | 265,000 | >1,000,000 |

### EXAMPLE 5

| | Formula A | Formula B | Formula C |
|---|---|---|---|
| Raw Material | % weight | % weight | % weight |
| Phase A | | | |
| Glyceryl Stearate Citrate | 4.0 | 4.0 | 4.0 |
| Stearyl Heptanoate, Stearyl | 3.0 | 3.0 | 3.0 |
| Caprylate | | | |
| Mineral oil | 7.0 | 7.0 | 7.0 |
| Stearyl Alcohol | 1.5 | 1.5 | 1.5 |
| Glyceryl Stearate | 1.5 | 1.5 | 1.5 |
| Dimthicone | 2.0 | 2.0 | 2.0 |
| Phase B | | | |
| Water | q.s 100 | q.s. 100 | q.s. 100 |
| Carbomer | 0.15 | 0.15 | 0.15 |
| 1,2-hexane diol and 1,2-octane | 0.50 | — | 0.50 |
| diol (1:1 w/w) | | | |
| Tropolone | — | 0.01 | 0.01 |
| Phase C | | | |
| Aminomethyl Propanol | 0.1 | 0.1 | 0.1 |

The challenge test results for the cosmetic formulations of Example 5 (all having a pH-value of 5.5) were as follows:

| | | | Formula C |
|---|---|---|---|
| | | | (0.5% 1,2-hexane |
| | Formula A | | Diol and 1,2- |
| | (0.5% wt 1,2-hexane | | Octane diol, 1:1 w/w |
| | diol and 1,2-octane | Formula B | and 0.01% |
| | diol; 1:1 w/w) | (0.01% tropolone) | tropolone |
| | Aspergillus niger | Aspergillus niger | Aspergillus niger |
| | [microbial count, | [microbial count, | [microbial count, |
| Days | CFU/ml] | CFU/ml] | CFU/ml] |
| 0 | 280000 | 280000 | 280000 |
| 2 | 80000 | 72000 | 300 |
| 7 | 68000 | 2000 | 0 |
| 14 | 40000 | 4800 | 0 |
| 28 | 30000 | 100 | 0 |

Now that the invention has been described, what is claimed is:

1. An antimicrobial composition, comprising an antimicrobial effective amount of
(a) at least one straight-chain 1,2-alkanediol, the chain length of which is in the range of 5 to 10 C-atoms, and
(b) at least one preservative selected from sorbate and its salts, parabenes, iodopropynyl butylcarbamate, ethylene diamene tetraacetic acid and its salts, tropolone compounds and sisquiterpenes.

2. The antimicrobial composition according to claim 1, said component (a) comprising at least two 1,2-alkanediols, wherein the proportions of the said diols in the mixture are set such that their antimicrobial action is synergistically intensified.

3. The antimicrobial composition according to claim 1, wherein the mixture contains 1,2-hexanediol and one, two or three further straight-chain 1,2-alkanediols, the chain length of which in each case is in the range of 5 to 10 C atoms.

4. The antimicrobial composition according to claim 1, wherein component (a) comprises:
1,2-hexanediol and 1,2-octanediol,
1,2-hexanediol and 1,2-decanediol,
1,2-pentanediol, 1,2-hexanediol and 1,2-octanediol,
1,2-hexanediol, 1,2-octanediol and 1,2-decanediol or
1,2-pentanediol, 1,2-hexanediol and 1,2-decanediol, the proportions of the said diols in the mixture being set such that their antimicrobial action is synergistically intensified.

5. The antimicrobial composition according to claim 1, wherein the at least one preservative is sorbate or its salts.

6. The antimicrobial composition according to claim 1, wherein the at least one preservative is selected from parabene, iodopropynyl butylcarbamate, and ethylene diamene tetraacetic acid and its salts.

7. The antimicrobial composition according to claim 1, wherein the at least one preservative is a tropolone compound selected from tropolone (2-hydroxycyclohepta-2,4,6-trienone) and thujaplicins (alpha-, beta- and/or gamma-thujaplicin).

8. The antimicrobial composition according to claim 1, wherein the at least one preservative is a sisquiterpene selected from the group consisting of bisabolol and farnesol.

9. An antimicrobial mixture comprising:
(a) 1,2-hexanediol and 1,2-octanediol and also optionally 1,2-pentanediol and/or 1,2-decanediol and also
(b) one, two or more substances selected from the group consisting of potassium sorbate, parabens and iodopropynyl butylcarbamate (IPBC).

10. An antimicrobial mixture according to claim 9, comprising 2-10 wt. % IPBC based on the total quantity of the 1,2-hexanediol and 1,2-octanediol and also optionally 1,2-pentanediol and/or 1,2-decanediol employed.

11. An antimicrobial mixture according to claim 10, comprising
(a) 1,2-hexanediol and 1,2-octanediol and also optionally 1,2-pentanediol and/or 1,2-decanediol and also
(b) one, two or more substances selected from the group consisting of potassium sorbate, parabens and iodopropynyl butylcarbamate (IPBC),
wherein the quantity of substances pertaining to group (a) and the quantity of substances pertaining to group (b), considered separately in each instance, is not antimicrobially effective, the total quantity of substances pertaining to the groups (a) and (b), however, is antimicrobially effective.

12. A cosmetic or pharmaceutical preparation or foodstuff comprising:
(a) 1,2-hexanediol and 1,2-octanediol and also optionally 1,2-pentanediol and/or 1,2-decanediol, and also
(b) one, two or more substances selected from the group consisting of potassium sorbate, parabens and iodopropynyl butylcarbamate (IPBC), and also
(c) further conventional constituents,
wherein the total quantity of substances pertaining to groups (a) and (b) lies within the range from 0.01 wt. % to 30 wt. %, relative to the total mass of the preparation or of the foodstuff.

13. A preparation or foodstuff according to claim 11, comprising 2-10 wt. % IPBC based on the total quantity of the 1,2-hexanediol and 1,2-octanediol and also optionally 1,2-pentanediol and/or 1,2-decanediol employed.

14. A preparation or foodstuff according to claim 11, comprising
(a) 1,2-hexanediol and 1,2-octanediol and also optionally 1,2-pentanediol and/or 1,2-decanediol and also
(b) one, two or more substances selected from the group consisting of potassium sorbate, parabens and iodopropynyl butylcarbamate (IPBC),
wherein the quantity of substances pertaining to group (a) and the quantity of substances pertaining to group (b), considered separately in each instance, is not antimicrobially effective, the total quantity of substances pertaining to the groups (a) and (b), however, is antimicrobially effective.

15. A process for the preservation or antimicrobial treatment of a perishable product, comprising:
contacting the perishable product with an antimicrobially effective quantity of a mixture comprising:
(a) 1,2-hexanediol and 1,2-octanediol and also optionally 1,2-pentanediol and/or 1,2-decanediol and also
(b) one, two or more substances selected from the group consisting of potassium sorbate, parabens and iodopropynyl butylcarbamate (IPBC).

16. The process as in claim 15, wherein said mixture is applied in an amount effective against Aspergillus niger.

17. A process for the cosmetic and/or therapeutic treatment of
(i) micro-organisms causing body odour,
(ii) micro-organisms causing acne and/or
(iii) micro-organisms causing mycoses,
comprising the topical application of an antimicrobially effective quantity of a mixture comprising:
(a) 1,2-hexanediol and 1,2-octanediol and also optionally 1,2-pentanediol and/or 1,2-decanediol and also
(b) one, two or more substances selected from the group consisting of potassium sorbate, parabens and iodopropynyl butylcarbamate (IPBC).
